(19) United States
(12) Patent Application Publication
(10) Pub. No.: US 2024/0304285 A1
(43) Pub. Date: Sep. 12, 2024

(54) System and Method for Prediction of Molecular Dynamics and Chemical Properties Using Equivariant Neural Operators

(71) Applicant: CARNEGIE MELLON UNIVERSITY, Pittsburgh, PA (US)

(72) Inventors: Xingping SHEN, Pittsburgh, PA (US); Venkatasubramanian VISWANATHAN, Pittsburgh, PA (US)

(73) Assignee: CARNEGIE MELLON UNIVERSITY, Pittsburgh, PA (US)

(21) Appl. No.: 18/552,362

(22) PCT Filed: Mar. 29, 2022
(86) PCT No.: PCT/US2022/022237

§ 371 Date: Sep. 25, 2023

Related U.S. Application Data
(60) Provisional application No. 63/234,768, filed on Aug. 19, 2021.
(60) Provisional application No. 63/167,255, filed on Mar. 29, 2021.

(51) Int. Cl. G16C 20/30; G16C 20/70

(52) U.S. Cl. CPC G16C 20/30; G16C 20/70

(57) ABSTRACT

Disclosed herein is a system and method using an equivariant neural network for predicting quantum mechanical charge density. The equivariant neural network serves as a surrogate for the density-functional theory used to calculate a selfconsistent field and predicts the central observable charge density, which, in addition to enabling force calculations, can also accelerate DFT itself and compute a full range of chemical properties.

### RELATED APPLICATIONS

This application claims the benefit of U.S. Provisional Patent Applications Nos. 63/167,255, filed Mar. 29, 2021, and 63/234,768, filed Aug. 19, 2021, the contents of which are incorporated herein in their entireties.

### GOVERNMENT INTEREST

This invention was made with the support of the U.S. Government under contract DEAR0001211, awarded by the U.S. Department of Energy. The U.S. Government has certain rights in this invention.

### BACKGROUND

Density Functional Theory (DFT) is a computational quantum mechanical modelling method that is a widely used to calculate the electronic structure of atoms, molecules, and solids. Its goal is the quantitative understanding of material properties from the fundamental laws of quantum mechanics. Additionally, it can be used to compute chemical properties and drive ab initio molecular dynamics. It computes the quantum mechanical observable electron density via a self-consistent field (SCF) procedure which, unfortunately, scales as O n with respect to the number of atoms. As such, it becomes computationally intractable for large systems of thousands or millions of atoms.

To remedy scalability while maintaining accuracy at the DFT level, machine learning inter-atomic potentials (MLIP) have been developed as molecular dynamics surrogates. MLIP predicts atomic forces in O n scale via a neural network. However, the method requires thousands of samples per molecule class and, most importantly, is restricted to forces and energy prediction, thus lacking the versatility of DFT.

### SUMMARY

Density-functional theory (DFT) is a widely used quantum-mechanical approach to model the electronic structure in matter from first principles. Based on DFT approaches, many chemical and physical properties can be accurately predicted, such that the suitability of a compound towards applications can be simulated before costly manufacture. An established and highly successful application of DFT is the discovery of novel materials by systematic high-throughput screening through a design space of candidate compounds. In such methods, a limiting factor is the cost of the required DFT simulations, with the self-consistent field (SCF) procedure being the most time-consuming component. In this procedure, a fixed-point problem for the electron density is solved starting from an initial guess, commonly built from precomputed atomic data.

Disclosed herein is a method using a neural-network model as a surrogate for the DFT to predict the converged fixed-point density by feeding the neural network a standard (cheaply computable) initial guess as well as the molecular structure.

The system and method disclosed herein uses an equivariant neural network for predicting quantum mechanical charge density that is then used for accelerating DFT calculations computing atomic forces in molecular dynamics and calculating chemical properties including, for example, multi-pole moments, band structure and phonon states.

The invention includes a complete DFT surrogate that predicts the central observable charge density, which, in addition to enabling force calculations, can also accelerate DFT itself and compute a full range of chemical properties.

The invention further introduces equivariant neural operators for learning resolution invariant as well as translation and rotation equivariant transformations between sets of tensor fields. Input and output may contain arbitrary mixes of scalar fields, vector fields, second order tensor fields and higher order fields. Tensor field convolution layers of the invention emulate any linear operator by learning its impulse response or Green's function as the convolution kernel. The tensor field attention layers emulate pairwise field coupling via local tensor products. Convolutions and associated adjoints can be in real or Fourier space allowing for linear scaling. By unifying concepts from E3NN, TBNN and FNO, the invention achieves good predictive performance on a wide range of PDEs and dynamical systems in engineering and quantum chemistry.

### BRIEF DESCRIPTION OF THE DRAWINGS

By way of example, a specific exemplary embodiment of the disclosed system and

method will now be described, with reference to the accompanying drawings, in which:

FIG. 1 is a simplified block diagram of the architecture of the disclosed system and method.

FIG. 2 is a block diagram of the architecture of the equivariant neural network used as the DFT surrogate.

FIG. 3 is an Illustration of rotational and translational equivariance.

### DETAILED DESCRIPTION

With reference to FIG. 1, the system and method described herein includes a charge density surrogate 100, a DFT engine 200, and a collection of properties calculators 300 relying on charge density. Surrogate 100 takes as input a set of atomic moieties and positions 10 and outputs the charge density distribution 190. Charge density distribution 190 may be used as input for the calculation of any one of a number of properties 300, including, for example, forces calculator 310 and multipole moments calculator 320. Charge density distribution 190 may also be input to DFT engine 200 as the initial charge density distribution for SCF calculations, producing chemical properties including, for example, band structure and phonon states.

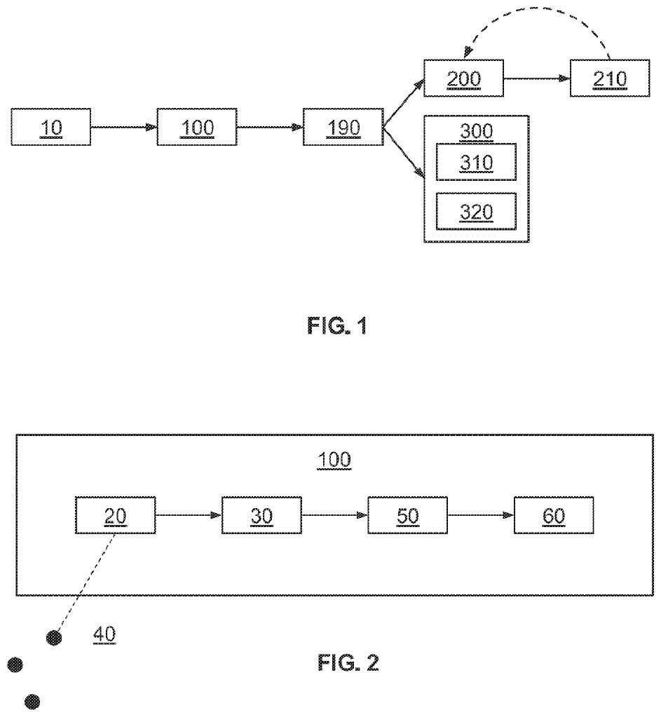

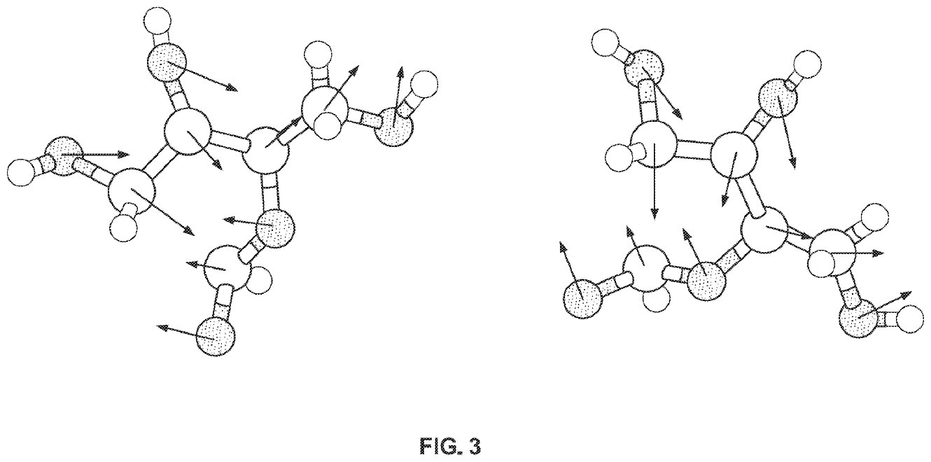

Surrogate 100 is shown in FIG. 2. In some embodiments, surrogate 100 is an equivariant neural network with intermediate tensor features 30 at each atomic position (node) 20. Equivariant refers to rotational equivariance and translational invariance, as illustrated in FIG. 3. Tensor features 30 (e.g., scalars, 3D vectors, quadrupoles . . . ) is akin to a local symmetry descriptor at each atomic position 20. In this case, the set of atomic moieties and positions 10 is encoded as an array of scalars 20 which depends on the atomic moiety. In subsequent layers, the set of tensor features 30 is computed at each node 40 via equivariant convolution. To compute density at any probe point 50 in space, the final tensors at each node are projected along the edge between 40 and 50 to produce a final encoding 60 at each 50. Final encoding 60 enters a dense scalar neural network to predict density.

In some embodiments, forces calculator 310 uses an Ewald scheme such as particle mesh to efficiently run in O n log n scale. In some embodiments, DFT engine 200 uses charge density distribution 190 to run at least one round of SCF to compute final charge density distribution 210 and estimated error in charge density distribution 190. Optionally, multiple rounds of SCF may also be run for DFT convergence by feeding final charge density distribution 210 back into DFT engine 200 multiple times. An accurate charge density distribution 190 predicted by the equivariant neural network reduces the total number of SCF iterations required for DFT convergence.

For surrogate 100, the invention unifies concepts from Euclidean neural networks (E3NN, previously known as “tensor field networks”), tensor basis neural networks (TBNN) and Fourier neural operators (FNO). E3NN generalizes convolutions to tensor features by defining appropriate tensor products over spherical harmonic projections. However, E3NN is a graph convolutional network, acting on features over a discrete point cloud. Further, as with CNNs, long range or domain wide convolutions scales poorly as O n. In contrast, the invention extends E3NN style convolutions to tensor fields defined over all of [symbol] represented as a regular grid. Further, O N log N scaling is achieved for long range convolutions by computing them in Fourier space via the convolution theorem in FNO. An equivariant attention layer for pairwise coupling between tensor features at a point is also implemented, which is akin to TBNN but realized via tensor products.

In a first embodiment of the invention, an equivariant operator is trained to directly map a scalar field of nuclear proton charges to a scalar field of electronic density. The equivariant operator F is needed between sets of tensor fields u and v over [symbol] . The input and output sets can, in general, be of different cardinalities and contain tensor fields of arbitrary and different ranks.

F is implemented as a composition of a tensor field convolution linear layer L, a local product bilinear layer A, and a local nonlinear layer N. Canonically, F N AL but different layers may be composed together as needed. All layers use tensor products and norm operations which are equivariant.

$$\begin{matrix} & Fu \text{?} v \\ s . t . FTu & TV ∀ translations and rotations T\end{matrix} \qquad ( 1 )$$

$$\text{?} \text{indicates text missing or illegible when filed}$$

In a second embodiment of the invention, an equivariant operator is trained to map the scalar field of electronic density to the scalar quantity of total energy. Because the correct density minimizes the energy, an initial guess density is used and then the density is iteratively refined via gradient descent. This direct minimization method constitutes an orbital free density functional theory variant running in O n scale.

Using the second embodiment, the external nuclear potential, mean field, and approximate exchange-correlation energies may be directly calculated, for example, via a local density approximation, leaving the trainable part of the operator to learn the deviation. The operator is trained to not only predict energy but also have its functional derivative approach the zero field with respect to density (e.g., using 2nd order automatic differentiation and backpropagation). The second embodiment is generally more accurate and robust than the first embodiment. The second embodiment may optionally use the first embodiment to calculate the initial density.

Linear Layer—Tensor Field Convolutions—The output of any linear translation invariant system can be written as the convolution of the input and a characteristic impulse response. The impulse response for an operator's inverse is known as the operator's Green's function. If the system is also rotationally equivariant, the impulse response must be a product of a scalar radial function and a spherical harmonic.

The tensor field convolution layer of the invention learns as its convolution filter kernel the impulse response of a linear equivariant system. Concretely, a convolution relating u to v needs a filter tensor field of an appropriate rank and a tensor product suitable to the ranks. Common tensor products are given below. They can be generalized for higher order tensor fields via Clebsch-Gordan products.

$$Lu wu * h \\ {w}_{ℝ} ur ⊗ \text{?} , \to hr r dr \\ r ⊗ , \to Y r r dr \\ wR \vert r \vert {u}_{ℝ} \qquad ( 2 )$$

$$⊗ , \to \begin{matrix} & if l 0 and l if \\ scaler product & l 0 and l l l \\ dot product & for \\ cross product & 1 , 1 \to 1 \\ traceless symmetric matrix vector product & for 2 , 1 \to 1\end{matrix} \qquad ( 3 )$$

$$1 for l 0 \\ Y r for l 1 \\ 3 rr 𝕀 for l 2 \qquad ( 4 )$$

$$\text{?} \text{indicates text missing or illegible when filed}$$

For example, consider the convolution mapping a charge distribution (scalar field, rank l 0) to the electric field (vector field l 1) via Gauss's Law. The filter is a vector field with radial function 1ᵣ and rank 1 spherical harmonic r. The tensor product is scalar vector multiplication.

Long-Ranged Tensor Convolutions in Fourier Space—Tensor field convolutions are numerically implemented as separate scalar convolutions of the component scalar fields. Short-ranged convolutions (e.g., differential operators) are done directly, while long-range convolutions are efficiently done in Fourier space via the convolution theorem. FFT allows N log N scaling in the latter. Let [symbol] denote the Fourier transform, e is the i basis of the result tensor field, u i is the i scalar field component of tensor field u, and C is the Clebsch-Gordan coefficient of the appropriate tensor product.

$$u * hC \text{?} um * hne , \\ ℱ \text{?} C \text{?} ℱ um ℱ hn \text{?} e , \qquad ( 5 )$$

$$\text{?} \text{indicates text missing or illegible when filed}$$

Pairwise Attention Layer: Tensor Field Local Product—At each point, an output tensor field is a linear combination of the appropriate pointwise tensor products between pairs of input tensor fields. A pair may contain the same input field twice or an identity scalar field (to facilitate recovering the identity transformation). A pair is dropped for an output field if there's no appropriate tensor product matching the ranks.

$$Au \text{?} rwu \text{?} {r}_{lua , lub \to lvj}^{⊗} {u}_{b} r , \qquad ( 6 )$$

$$\text{?} \text{indicates text missing or illegible when filed}$$

Nonlinear Layer: Local Norm Nonlinearity—Nonlinear activations are local and pointwise. It rescales a tensor field as a function of the field's norm at each point. The function can be an affine followed by ReLU. Because norms are rotation invariant, overall equivariance is preserved.

$$Nu \text{?} r σ a u \text{?} rbu \text{?} r \qquad ( 7 )$$

$$\text{?} \text{indicates text missing or illegible when filed}$$

Neural Network Parameters—F can be learned by parameterizing it for pairs of training data u, v. A few parameters are a, b, w while most parameters encode different radial functions of the tensor convolutions. For the latter, one scheme is sum of Gaussians, decay power laws, and derivative operators. Alternately, a neural network with r as input may be used.

$$R \text{?} rAe / Br \vert \text{?} \vert C \partial δ r \qquad ( 8 )$$

$$\text{?} \text{indicates text missing or illegible when filed}$$

The network is trained on data generated by PDEs and other physical problems including quantum chemistry. Almost perfect fits are achieved on linear PDEs (e.g., Poisson's equation, Gauss's law and Stokes flow). This is unsurprising as the tensor field convolutions are tantamount to learning Green's functions which completely characterize linear systems. Almost perfect fits are also achieved for some nonlinear PDE functions with pairwise coupling such as the advection time derivative. Tensor convolutions can encompass differential operators while local tensor products in equivariant attention represent coupling. Good, but not as perfect performance is achieved on the general nonlinear quantum mechanics problem of predicting electronic densities from nuclei positions. Performance should improve from stacking multiple linear and nonlinear layers.

To generate data, time independent PDEs are solved using FDM Green's functions (for Poisson's, Gauss's, Stokes flow). Time dependent PDEs (diffusion advection) have their time derivative directly evaluated as the output. In all cases, a 16×16×16 domain is used. Input scalar and vector fields are initiated randomly per voxel while their values outside of the domain are implicitly set to 0.

As would be realized by one of skill in the art, the disclosed method described herein can be implemented by a system comprising a processor and memory, storing software that, when executed by the processor, performs the functions comprising the method.

As would further be realized by one of skill in the art, many variations on implementations discussed herein which fall within the scope of the invention are possible. Moreover, it is to be understood that the features of the various embodiments described herein were not mutually exclusive and can exist in various combinations and permutations, even if such combinations or permutations were not made express herein, without departing from the spirit and scope of the invention. Accordingly, the method and apparatus disclosed herein are not to be taken as limitations on the invention but as an illustration thereof. The scope of the invention is defined by the claims which follow.

1. A method comprising:
inputting a set of atomic moieties and positions to a neural network; receiving, from the neural network, an initial charge density distribution; and inputting the initial charge density distribution to a density functional theory engine to calculate a self-consistent field.

2. The method of claim 1 further comprising:
repeatedly inputting the self-consistent field to the density functional theory engine until density functional theory convergence is achieved.

3. The method of claim 1 wherein the set of atomic moieties and positions is in the form of an array of scalars dependent upon the atomic moiety.

4. The method of claim 1 wherein the neural network is an equivariant neural network outputting a set of tensor features representing the charge density distribution at each atomic position.

5. The method of claim 4 wherein the set of tensor features is computed at each atomic position via an equivariant convolution.

6. The method of claim 3 wherein the equivariant neural network is equivariant in terms of rotation and translation.

7. The method of claim 5 further comprising inputting the set of tensor features to a scaler neural network to predict charge density at each atomic position.

8. The method of claim 1 further comprising
inputting the charge density distribution produced by the equivariant neural network to one or more property calculators.

9. The method of claim 8 wherein the one or more property calculators include a forces calculator and a multipole moments calculator.

10. The method of claim 6 wherein the equivariant neural network implements an equivariant operator operating between sets of tensor fields.

11. The method of claim 10 wherein the equivariant operator is implemented as a composition of a tensor field convolution linear layer, a local product by linear layer and a local nonlinear layer.

12. The method of claim 11 wherein the output of the equivariant neural network is a convolution of the input and a characteristic impulse response.

13. The method of claim 12 wherein the impulse response is a product of a scaler radial function and a spherical harmonic, such that the equivariant neural network is rotationally equivariant.

14. The method of claim 6 wherein the equivariant operator maps a scalar field of electronic density to a scalar quantity of total energy.

15. The method of claim 14 further comprising:
iteratively refining the initial charge density distribution via gradient descent.

16. The method of claim 15 wherein external nuclear potential, mean field, and approximate exchange-correlation energies are directly calculated.

17. The method of claim 16 wherein a trainable portion of the equivariant operator learns a deviation of the gradient descent.

18. The method of claim 17 wherein the operator is trained to predict energy and have its functional derivative approach a zero field with respect to charge density distribution.

19. A system comprising:
a processor; and software, which, when executed on the processor, causes the system to perform the functions of:
inputting a set of atomic moieties and positions to an equivariant neural network; receiving, from the equivariant neural network, an initial charge density distribution; and inputting the initial charge density distribution to a density functional theory engine to calculate a self-consistent field.

20. The system of claim 19 the software further causing the system to perform the functions of: repeatedly inputting the self-consistent field to the density functional theory engine until density functional theory convergence is achieved.

21. The system of claim 19 wherein the set of atomic moieties and positions is in the form of an array of scalars dependent upon the atomic moiety, and further wherein the equivariant neural network outputs a set of tensor features representing the charge density distribution at each atomic position.

22. The system of claim 19 wherein the equivariant neural network is equivariant in terms of rotation and translation.

23. The system of claim 19, the software further causing the system to:
input the charge density distribution produced by the equivariant neural network to one or more property calculators, the one or more property calculus including a forces calculator and a multipole moments calculator.

24. The system of claim 19 wherein the equivariant neural network implements an equivariant operator operating between sets of tensor fields.

25. The system of claim 24 wherein the equivariant operator is implemented as a composition of a tensor field convolution linear layer, a local product by linear layer and a local nonlinear layer.

26. The system of claim 24 wherein the equivariant operator maps a scalar field of electronic density to a scalar quantity of total energy.
